(19) United States
(12) Patent Application Publication
(10) Pub. No.: US 2024/0058570 A1
(43) Pub. Date: Feb. 22, 2024

(54) DILATOR LUMEN CHANNELS FOR COAXIAL FLUID TRANSFER

(71) Applicant: BOSTON SCIENTIFIC MEDICAL DEVICE LIMITED, Galway (IE)

(72) Inventor: John Paul Urbanski, Toronto (CA)

(21) Appl. No.: 18/366,015

(22) Filed: Aug. 7, 2023

Related U.S. Application Data
(60) Provisional application No. 63/371,555, filed on Aug. 16, 2022.

(51) Int. Cl. A61M 25/00; A61B 18/14; A61M 5/00; A61M 29/00

(52) U.S. Cl. CPC A61M 25/0026; A61B 18/1492; A61M 5/007; A61M 29/00; A61B 2017/00247

(57) ABSTRACT

An apparatus and kit for coaxial fluid transfer within a tubular member that forms a dilator. One or more channels are provided that form a fluid path through the dilator irrespective of the presence of an elongated device such as a guidewire, a wire-based needle, a radiofrequency-based puncture device, a mechanical-based puncture device, a catheter mechanism, a stylet mechanism, or any suitable device dimensioned for insertion into a dilator for accessing tissue of a patient. The channels may be located on a lumen surface, within the distal tip of the tubular member, or may extend a partial or full length of the tubular member.

### CROSS REFERENCE TO RELATED APPLICATIONS

This application claims the benefit of priority to U.S. Provisional Application Ser. No. 63/371,555, titled “DILATOR LUMEN CHANNELS FOR COAXIAL FLUID TRANSFER,” filed Aug. 16, 2022, the entire disclosure of which is incorporated herein by reference in its entirety.

### FIELD OF THE INVENTION

The present invention relates generally to an apparatus usable within the body of a patient. More specifically, the present invention relates to fluid transfer within medical devices that use a dilator and optional sheath in conjunction with puncturing tissue.

### BACKGROUND OF THE INVENTION

In the field of surgical apparatus usable within the body of a patient, there exists medical devices that include dilators with and without sheaths in conjunction with puncturing tissue. The target site may include a tissue within the heart of a patient, for example, the atrial septum of the heart. In such situations, the target site may be accessed via the inferior vena cava (IVC), for example, through the femoral vein. In particular, transseptal puncture is a procedure commonly performed when a physician accesses left-sided chambers of the heart such as the left atrium or left ventricle. Venous vasculature is accessed percutaneously, and a catheter is used to facilitate delivery of a puncture device from the percutaneous access site to the fossa ovalis in the right atrium 207 such as shown in FIG. 2. The fossa ovalis 200 is a thin flap of tissue that covers the foramen ovale which is a remnant of the fetal heart and an optimal site for puncture via a puncture device such as an electrode 103.

Once an initial puncture hole in the tissue has been created by the puncture device, the puncture hole must be expanded by way of a dilator to allow for the delivery of a therapy sheath through this site. As therapies continue to evolve in their complexity and capabilities, progressively larger therapy sheaths are required to cross the puncture hole made in the fossa ovalis. Known methods of expanding a puncture hole include the use of dilators that have a tapered distal end that progressively expands a puncture hole as the dilator is advanced across the fossa ovalis. Known dilators have larger outer diameters so that the puncture hole is expanded further to accommodate therapy sheaths with larger outer diameters.

Fluid transfer via a lumen within the puncture device, the dilator, and/or the sheath is also known in the field of surgical apparatus. Fluid may travel in either direction, from or to the puncture site, within such lumen(s). One such known arrangement is shown by way of FIG. 1. Here, a known medical device 100 is illustrated that includes an electrode 103 as a puncture device of the known radiofrequency (RF) type though mechanical puncture devices such as a conventional Brockenbrough transseptal needle may be used instead of an electrode. The medical device 100 typically includes a hub 113 at a proximal region 114, a curved or shapeable section 102 at a distal region 101. In terms of an electrode-based medical device, the overall system would typically further include an RF generator 106 with a related grounding pad 104 with appropriate electrical connections via lead wires 105. Fluid may be passed through, or extracted from, the medical device 100 via the lumen(s) of the puncture device, dilator, and/or sheath by way of tubing 112 by a syringe 109 connected to the tubing 112 at adapter fitting 111. Alternatively, the adapter fitting 111 may be provided to attach with an identical fitting associated with tubing 110 connected to a pressure sensing device 108 and a pressure monitoring system 107. When in place within a patient during use such as shown in FIG. 2, continuity of fluid from an opening at the distal region 101 through the hub 113 and tubing 110, 111 thus enables such pressure sensing as is known in the medical art.

In addition to pressure sensing, the arrangement shown in FIG. 1 and FIG. 2 allows a physician to access the right atrium 207, for example, through the inferior vena cava 206 whereby the distal region 101 of the medical device may abut the fossa ovalis 200. With regard to FIG. 2 as shown in close-up, a puncturing device 205 is shown within a dilator 202. An aperture 204 within the puncturing device itself may be provided if such puncturing device includes a lumen. The aperture 204 enables fluid flow 203 (e.g., contrast dye for tissue staining or procedure-specific medication) to be applied to the patient's septal tissue via the dilator lumen 201. However, it should be noted that the annular space within the dilator lumen 201 is reduced by the presence therein of the structure that forms the puncturing device. This reduction of fluid flow within such annular space hinders the ability, for example, of adequate application of contrast dye. This is especially true when, as is typical, the tip of a dilator includes a narrowed portion of lumen as shown in FIG. 3A and FIG. 3B.

With reference to FIG. 3A and FIG. 3B, a close-up illustration shows a cross-section of a known dilator 300 revealing a known puncturing device 205 and electrode 103 located at the narrowed proximal tip of the dilator. Because the face of the dilator's distal tip 304 has a relatively small surface area and the outer circumference of the dilator 300 tapers towards the dilator's distal tip 304 as shown, the lumen forms a constricted zone 302 that results in reduced fluid flow 303 when the electrode 103 is located at the proximal tip. Thus, fluid flow 301 within the dilator lumen 305 is disadvantageously restricted.

While some known transseptal needles include a lumen that facilitates injection of contrast fluid or aspiration of blood for pressure traces directly at the tip of the elongate device itself, the lack of a lumen in elongated devices such as guidewire-based puncture devices as well as the tight fit that all these devices maintain inside of dilators necessitates an alternative manner of delivering fluid to the distal end of these dilator/elongated device systems.

What is needed therefore is a dilator that resolves the deficiencies of prior medical systems.

### SUMMARY OF THE INVENTION

In a first broad aspect, embodiments of the present invention include an apparatus for fluid transfer, the apparatus having a tubular member having a circumferential sidewall, the circumferential sidewall including a tapered portion with an outer diameter that is tapered towards a distal tip of the tubular member; a lumen for fluid transfer through the distal tip, the lumen centrally located within the circumferential sidewall; and a channel coaxial with the lumen and located at least at a distal region of the tubular member; wherein a fluid is movable through the channel irrespective of the presence of an elongated device located adjacent the channel.

As features of the first broad aspect the channel may be located along a tapered portion of the tubular member, and the apparatus may include at least one additional channel. Each channel may be separated from one another by an arc length around a periphery of the lumen. The arc length separating each channel may be equidistant. The lumen may be at least partially occluded by the elongated device located adjacent the channel. The channel may extend between a hub connected to the tubular member and the distal tip of the tubular member, and the channels may be open to the lumen.

Some embodiments disclose the tubular member may be a dilator and the elongated device may be wires that form a guidewire, a wire-based needle, an RF needle (i.e., radiofrequency-based puncture device), a mechanical needle (i.e., mechanical-based puncture device), a catheter mechanism, a stylet mechanism, or any suitable device dimensioned for insertion into a dilator for accessing tissue of a patient.

In a second broad aspect embodiments of the present invention include an apparatus that may include an inner tubular member forming a dilator that is concentric to the tubular member which forms a sheath around the dilator, where the lumen is a sheath lumen.

Some embodiments of the second broad aspect disclose that the channels may be formed within a distal section of the dilator. The channels may include a proximal port in fluid communication with the sheath lumen and a distal port located at the distal tip of the dilator. An interface between the sheath and the dilator may form a seal by an interference fit, and fluid transfer through the channels may be selectively enabled and disabled by relative linear movement between the sheath and the dilator that places the proximal port of each channel, respectively, into and out of fluid communication with the sheath lumen.

In a third broad aspect, embodiments of the present invention include a kit forming an apparatus for fluid transfer, the kit including: an elongated device capable of insertion into a tubular member for accessing tissue of a patient; and a tubular member capable of accepting the elongated device, the tubular member having a circumferential sidewall, the circumferential sidewall including a tapered portion with an outer diameter that is tapered towards a distal tip of the tubular member; a lumen capable of fluid transfer through the distal tip, the lumen centrally located within the circumferential sidewall; and a channel coaxial with the lumen and located at least at a distal region of the tubular member; wherein, upon insertion of the elongated device into the tubular member, a fluid is capable of movement through the channel irrespective of the presence of the elongated device located adjacent the channel. The tubular member may form a dilator including at least one additional channel.

Some embodiments of the second broad aspect disclose that the kit may include an inner tubular member capable of forming a dilator that is concentric to the tubular member, wherein the tubular member forms a sheath around the dilator, the lumen is a sheath lumen, and the dilator includes at least one additional channel. Each of the channels may be formed within a distal section of the dilator, and each of the channels may include a proximal port capable of fluid communication with the sheath lumen and a distal port located at the distal tip of the dilator. An interface may be provided between the sheath and the dilator that is capable of forming a seal by an interference fit, and fluid transfer through the channels is capable of being selectively enabled and disabled by relative linear movement between the sheath and the dilator that places the proximal port of each channel, respectively, into and out of fluid communication with the sheath lumen.

Example 1 is an apparatus for fluid transfer. The apparatus includes a tubular member having a circumferential sidewall. The circumferential sidewall includes a tapered portion with an outer diameter that is tapered towards a distal tip of the tubular member. The tubular member includes a lumen for fluid transfer through the distal tip. The lumen is centrally located within the circumferential sidewall. The tubular member includes a channel coaxial with the lumen and located at least at a distal region of the tubular member. A fluid is movable through the channel irrespective of the presence of an elongated device located adjacent the channel.

Example 2 is the apparatus of Example 1, wherein the channel is located along a tapered portion of the tubular member.

Example 3 is the apparatus of Example 2, further including at least one additional channel.

Example 4 is the apparatus of Example 3, wherein each channel is separated from one another by an arc length around a periphery of the lumen.

Example 5 is the apparatus of Example 4, wherein the arc length separating each channel is equidistant.

Example 6 is the apparatus of Example 2, wherein the lumen is at least partially occluded by the elongated device located adjacent the channel.

Example 7 is the apparatus of Example 6, wherein the channel extends between a hub connected to the tubular member and the distal tip of the tubular member.

Example 8 is the apparatus of Example 7, wherein the tubular member is a dilator.

Example 9 is the apparatus of Example 8, wherein the channel is open to the lumen.

Example 10 is the apparatus of Example 3, further including an inner tubular member forming a dilator that is concentric to the tubular member which forms a sheath around the dilator, and the lumen is a sheath lumen.

Example 11 is the apparatus of Example 10, wherein the channels are formed within a distal section of the dilator.

Example 12 is the apparatus of Example 11, wherein each of the channels includes a proximal port in fluid communication with the sheath lumen and a distal port located at the distal tip of the dilator.

Example 13 the apparatus of Example 12, wherein an interface between the sheath and the dilator forms seal by an interference fit.

Example 14 is the apparatus of Example 13, wherein fluid transfer through the channels is selectively enabled and disabled by relative linear movement between the sheath and the dilator that places the proximal port of each channel, respectively, into and out of fluid communication with the sheath lumen.

Example 15 is a kit forming an apparatus for fluid transfer. The kit includes an elongated device capable of insertion into a tubular member for accessing tissue of a patient. The kit includes a tubular member capable of accepting the elongated device. The tubular member has a circumferential sidewall. The circumferential sidewall includes a tapered portion with an outer diameter that is tapered towards a distal tip of the tubular member. The tubular member has a lumen capable of fluid transfer through the distal tip. The lumen is centrally located within the circumferential sidewall. The tubular member has a channel coaxial with the lumen and located at least at a distal region of the tubular member. Upon insertion of the elongated device into the tubular member, a fluid is capable of movement through the channel irrespective of the presence of the elongated device located adjacent the channel.

Example 16 is the kit of Example 15, wherein the tubular member forms a dilator including at least one additional channel.

Example 17 is the kit of Example 15, further including an inner tubular member capable of forming a dilator that is concentric to the tubular member, wherein the tubular member forms a sheath around the dilator, the lumen is a sheath lumen, and the dilator includes at least one additional channel.

Example 18 is the kit of Example 17, wherein the channels are formed within a distal section of the dilator, and the channels each includes a proximal port capable of fluid communication with the sheath lumen and a distal port located at the distal tip of the dilator.

Example 19 is the kit of Example 18, wherein an interface between the sheath and the dilator is capable of forming a seal by an interference fit.

Example 20 is the kit of Example 19, wherein fluid transfer through the channels is capable of being selectively enabled and disabled by relative linear movement between the sheath and the dilator that places the proximal port of each channel, respectively, into and out of fluid communication with the sheath lumen.

### BRIEF DESCRIPTION OF THE DRAWINGS

In order that the invention may be readily understood, embodiments of the invention are illustrated by way of examples in the accompanying drawings.

FIG. 1 is a schematic of a known medical device and related system.

FIG. 2 illustrates partially cut-away views of the medical device of FIG. 1 in use during a medical procedure.

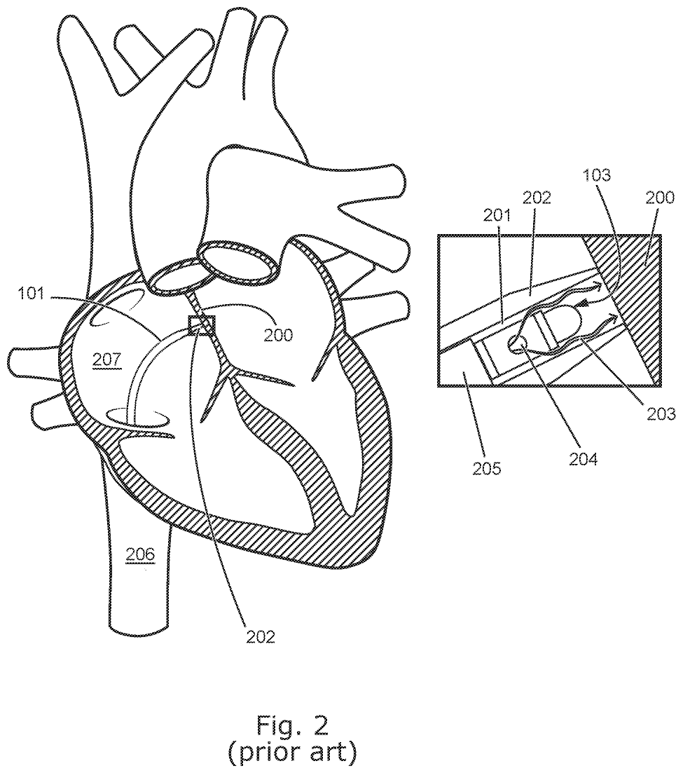

FIGS. 3A and 3B illustrate partially cut-away view and an end view, respectively, of an alternative known dilator and puncturing device that may be used in the system of FIG. 1.

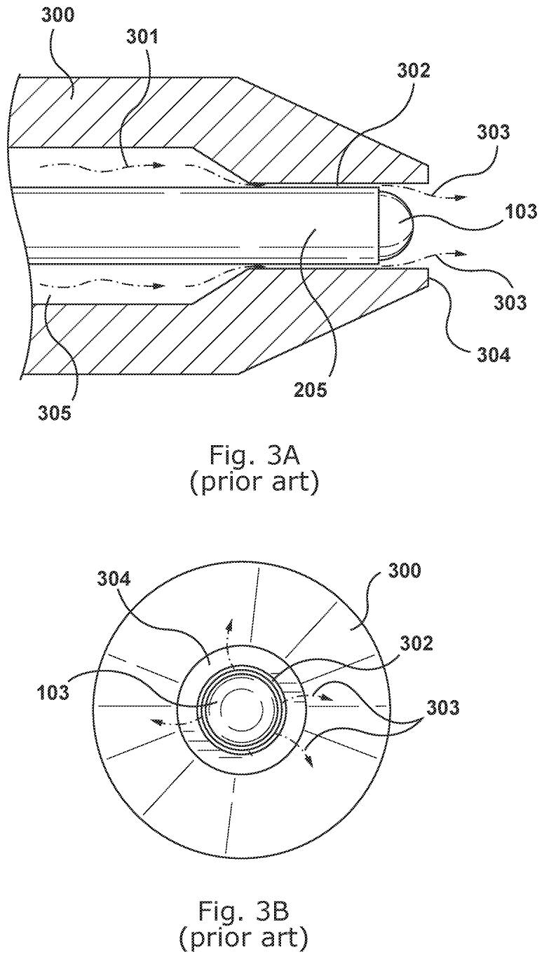

FIGS. 4A and 4B illustrate a partially cut-away side view and an end view, respectively, of a distal end of a dilator in accordance with an embodiment of the present invention utilizing one possible elongated device located therein.

FIGS. 5A and 5B illustrate a partially cut-away side view and an end view, respectively, of a distal end of a dilator in accordance with another embodiment of the present invention utilizing one possible elongated device located therein.

FIG. 6 illustrates a cross-sectional side view of a distal end of a dilator in accordance with a further embodiment of the present invention including an outer sheath and an elongated device located therein.

### DETAILED DESCRIPTION

With specific reference now to the drawings in detail, it is stressed that the particulars shown are by way of example and for purposes of illustrative discussion of embodiments of the present invention only. In this regard, no attempt is made to show structural details of the invention in more detail than is necessary for a fundamental understanding of the invention. The description taken with the drawings will make apparent to those skilled in the art how the several aspects of the invention may be embodied in practice.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As used herein, the terms ‘proximal’ and ‘distal’ are defined with respect to the user (e.g., a physician). That is, the term ‘proximal’ refers to a part or portion closer to the user, and the term ‘distal’ refers to a part or portion further away from the user when the device is in use on a subject (e.g., a patient). Also, it should be noted that while, for clarity of explanation, the term tubular or tubular member is used to describe the members that enclose elongated devices, the term tubular member is intended to describe both circular and non-circular embodiments of the enclosing member. The term tubular member is used in this disclosure to describe dilators, sheaths, and other members that define a lumen for containing an elongated device.

Elongated devices may include devices used for puncturing tissue, such as the septal tissue of a patient's heart, are typically either mechanical or electrosurgical in nature. Usually, such elongated devices are inserted into a patient's body through tubular members such as dilators or sheaths. The present invention utilizes existing puncturing devices and/or other elongated devices such as, but not limited to, guidewires or the like. The present invention also provides embodiments additionally utilizing sheaths.

It is common for elongated devices and corresponding tubular members to have a small gap between the outer diameter of elongated device and the inner diameter of an adjacent tubular member. Completely eliminating the gap would result in increased friction between the elongated device and the tubular member and would result in difficulty advancing the elongated device through the tubular member. In common arrangements, the gap is small enough to prevent a substantial flow of fluids such as contrast fluids, which are typically 3 to 5 times more viscous than water. The present invention avoids problems inherent to such common arrangements.

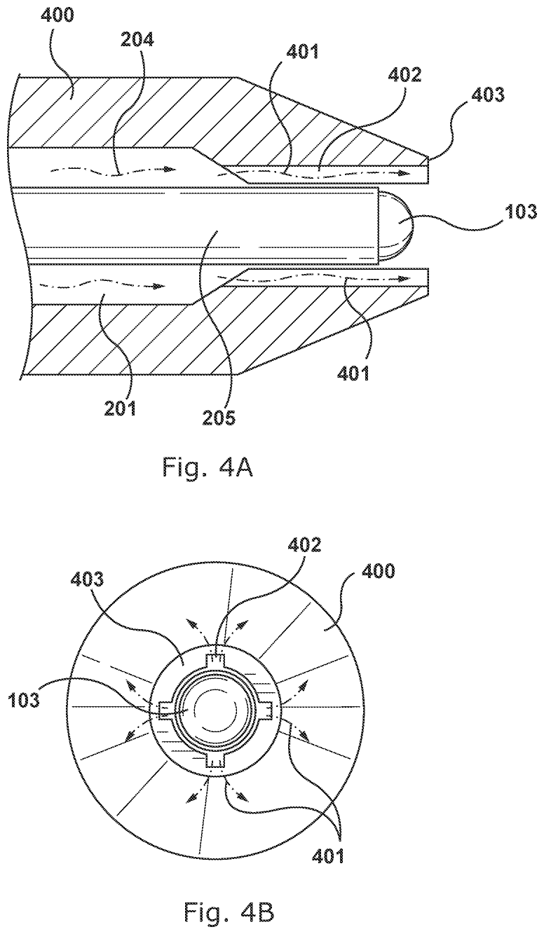

With reference to FIG. 4A, one possible embodiment of the present invention is shown in a partially cut-away side view. Here, a dilator 400 provides a dilator lumen 201 where fluid flow 204 may occur within the annular space that surrounds an elongated device in the form of a puncturing device 205 having an electrode 103 and situated within the dilator 400. As noted above, the invention may be used for a variety of applications including injecting fluid, withdrawing fluid, and measuring pressure. For example, delivery of contrast fluid may be provided through the distal end of the dilator lumen 201. While the flow is shown in one direction exiting the distal end of the dilator, it should be readily apparent that flow may occur in the opposite direction without straying from the intended scope of the present invention.

It should also be understood that the puncturing device 205 with an electrode 103 is a closed-end elongated device which may or may not have a lumen therein. In other words, the particular type of elongated device may vary according to the given medical procedure and related instrumentation. In all such variations, the present invention provides a new and useful dilator 400 irrespective of the specific elongated device maintained within the dilator lumen 201.

In order to advantageously increase flow rate through the tip of the dilator 400 where fluid exits (or enters) at the dilator end face 403, the present invention provides one or more channels 402. As further shown with additional reference to the end-view in FIG. 4B, such embodiment of the dilator 400 may include four channels 402 provided in a radial formation and located equidistant from one another where channel flow 401 occurs. However, any number of channels may be provided in any configuration (e.g., equidistantly placed, paired in groups, singular, etc.) and/or any shape (e.g., squared off, U-shaped, semi-circular, extended oblong-shaped, etc.) Moreover, the channels 402 are formed by any suitable manufacturing method to ensure an unimpeded micro-passage through the area between the inner sidewalls of the dilator tip and the puncturing device 205 that would otherwise be typically constricted in terms of fluid flow. It should further be understood that channel formation during manufacture removes material from the dilator sidewall such that thinning of the sidewall may occur. In such instances where thinning of the sidewall may be considered to affect the structural integrity of the dilator, the manufacturing process may therefore include adding material to the sidewall such as thickening the outer dilator surface or otherwise reinforcing the sidewall in a suitable manner to ameliorate any deleterious effects on structural integrity that may occur upon creation of the channel(s).

While puncturing device 205 with an electrode 103 is shown, it should be understood that any elongated devices may be used in conjunction with the inventive dilator structure such as, but not limited to, mechanical puncturing devices, open-lumen or closed-lumen elongated devices, guidewires, or elongated devices that are used with a transseptal sheath and dilator set such as in the form of a kit. Common to all arrangements is eliminating the problematic reduced flow at a constricted area of the dilator lumen located at the distal-most end of the dilator tip within which the elongated device(s) occupies.

By providing channels within such constricted area, the problem of hindered or ineffective flow of fluid at the tip of the dilator is avoided thereby increasing desired injection or withdrawal of fluid or enhancing pressure sensing by increasing continuity of fluid from the dilator tip to any pressure sensor in an overall system utilizing the present invention. This may be particularly beneficial when elongated devices such as RF guidewires are being used as transseptal needles for transseptal puncture procedures. Moreover, the present invention is effective in any elongated devices that lack a lumen such as guidewire-based puncture devices and compensates for the tight fit these devices maintain inside of dilators thereby providing an alternative manner of delivering fluid through the distal end of dilator/elongated device systems.

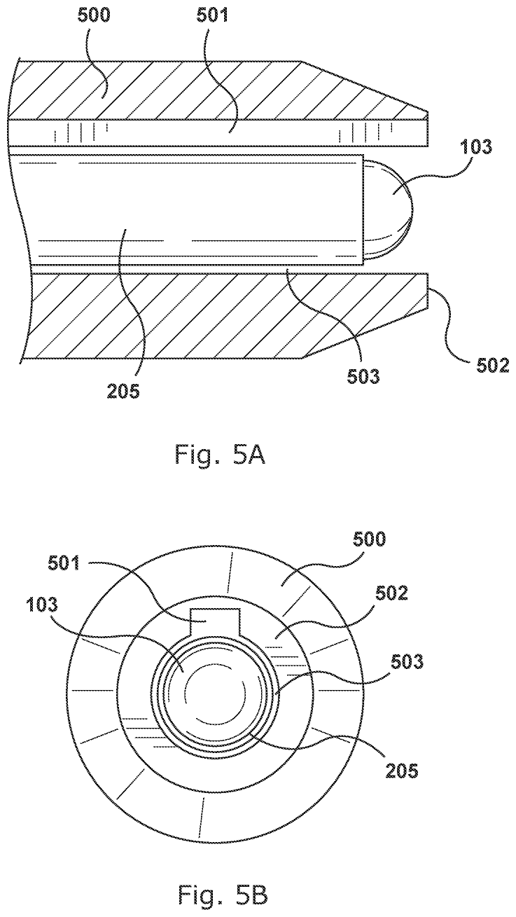

Having now shown and described an embodiment of the invention having multiple channels located at the distal tip, another embodiment will be discussed with regard to FIG. 5A and FIG. 5B. As an alternative to the multi-channel embodiment previously described above, FIG. 5A shows a dilator 500 having a single channel 501. Such configuration is advantageous in situations when the puncturing device 205 is sized relative to the dilator lumen 503 in such a way that the fit tolerance therebetween is relatively tight. In such configuration, it should be understood that the position of the puncturing device 205 and electrode 103 within the dilator 500 does not alter the annular space available for fluid flow through the dilator lumen 503. In other words, the dilator tip is not the only area where hindered fluid flow is improved by way of the present invention. Rather, the entirety of the length of the dilator lumen 503 is constricted as to fluid flow therethrough. However, the present invention provides the channel 501 along the length of the dilator 500 in order to provide a lengthwise path of unrestricted fluid flow between each end of the dilator lumen 503.

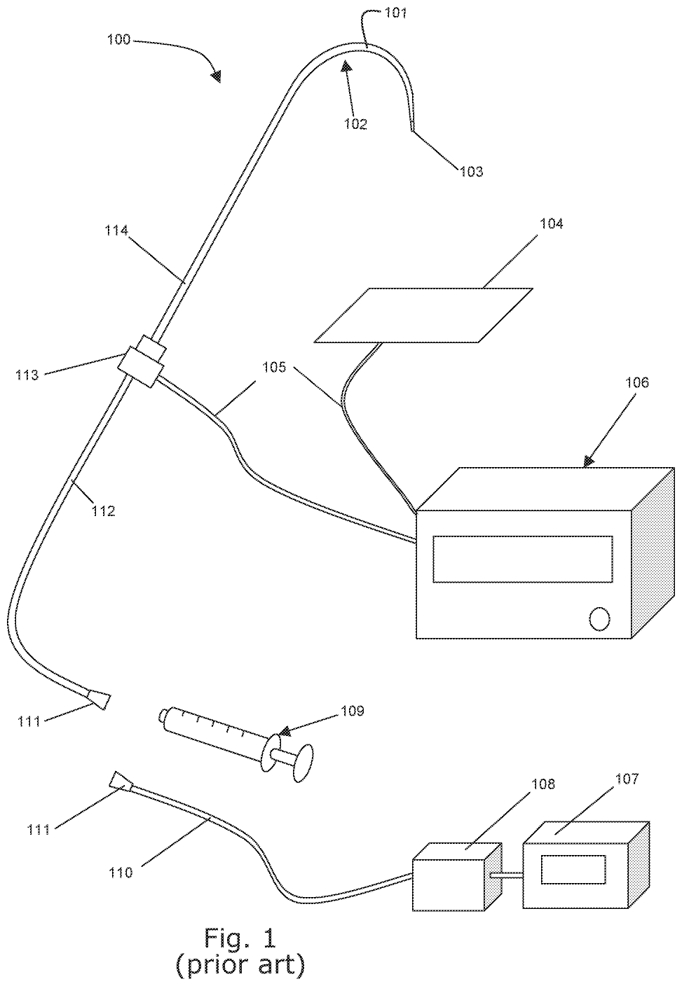

For illustrative clarity, FIG. 5A only shows a small portion of the dilator 500. However, it should be understood that the channel 501 may be present from the dilator's distal tip 502 to a hub when the present invention is used, for example, within a system such as shown in FIG. 1. In such instance, the channel 501 may provide a direct bypass pathway between a hub and the distal tip 502. While a squared off channel 501 is shown in FIG. 5B, the shape of channel 501 may be formed in any shape (e.g., squared off, U-shaped, semi-circular, extended oblong-shaped, etc.) without straying from the intended scope of the present invention.

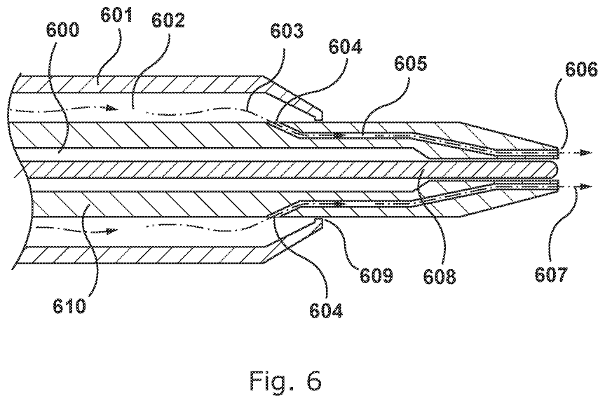

An additional embodiment of the present invention is shown and described by way of FIG. 6. Here, a cross-sectional side view of a distal end of a dilator 610 in accordance with yet still a further embodiment of the present invention is configured with an outer sheath 601 and an elongated device 608 located therein. The elongated device 608 is a tubular member that may include a dilator, a sheath, or some other member defining a lumen configured to receive a medical device. The elongated device 608 may be a relatively simple structure such as a guidewire as is illustrated or may be any device including, but not limited to, an RF or mechanical puncturing device, or any open-lumen or closed-lumen elongated device known in the medical art. As shown, the elongated device 608 is concentrically enveloped within nested tubular members that may include a transseptal sheath and dilator set such as in the form of a kit used together in the context of transseptal access procedures.

As mentioned, the sheath 601 and dilator 610 may be provided in a kit format and configured for use together. Moreover, the inner diameter of the sheath 601 is suitably dimensioned relative to the outer diameter of the dilator 610 such that a tight interference fit may exist at the distal end of the sheath 601 where contact is made with the dilator 610 thereby forming a ring seal 609. This aids in preventing fluid leakage from the annular space formed by the sheath lumen 602 that circumscribes the outer surface of the dilator 610.

In the embodiment shown in FIG. 6, the dilator 610 includes one or more channels 605 that form closed passageways that are internal to the structure forming the dilator 610 and located toward the distal tip of the dilator 610. These channels 605 are effectively internal conduits for flow of fluid (as indicated by dashed line arrows 603, 607). First port(s) 604 and second port(s) 606 form the entry and exit points, respectively, for fluid 603 flowing within the sheath lumen 602 and exiting as emitted fluid 607. However, should the flow be reversed from that illustrated (such as during any medical procedure extracting fluid (e.g., blood) from a patient), the second port 606 would of course be considered the entry point while the first port 604 would then be the exit point.

In operation, the embodiment of the present invention shown in FIG. 6 would function to allow passage of fluid between the sheath lumen 602 and the dilator distal tip 606 so long as the first port 604 remains in a proximal position relative to the seal 609. If one or both of the sheath 601 and the dilator 610 are slidingly moved in opposite directions relative to one another, it should be readily apparent that the first port 604 will move into and out of fluid communication with the fluid flow 603 within the sheath lumen 602. In such manner, emitted fluid 607 may be controlled to start and stop depending upon the location of the first port 604. Such control of the flow of fluid may of course be accomplished in the same manner during fluid extraction from a patient. In any such situation, it should be understood that the position of the elongated device 608 in this embodiment does not affect fluid flow through channels 605.

While two channels 605 are shown in FIG. 6, it should be further evident that any suitable number of channels may be provided to ensure adequate flow through the dilator 610. Likewise, the specific shape and route through the distal region of the dilator 610 may vary according to manufacturing methods and materials used without straying from the intended scope of the invention.

In all embodiments of the present invention, the distal tip of the dilator is substantially atraumatic. In other words, the distal tip including openings of the channels 401, 501, or the first and second ports 604, 606 are structured such that all edges exposed to tissue are substantially atraumatic, or blunt. As used herein, the terms ‘atraumatic’ and ‘blunt’ refer to a structure that is not sharp, and includes structures that are rounded, obtuse, or flat, amongst others. In embodiments wherein the distal tip of the inventive dilator is substantially blunt, the blunt distal end is beneficial for avoiding unwanted damage to non-target areas within the body. That is, if mechanical force is unintentionally applied to a medical device (such as within a medical device 100 shown in FIG. 1) incorporating the present inventive dilator when the distal end of the medical device is located at a non-target tissue, the medical device is less likely to perforate the non-target tissue.

In all embodiments of the present invention, at least some of the length of the channel(s) has a constant cross-sectional configuration which reduces turbulence and facilitates laminar flow, which in turn facilitates fluid movement.

In some embodiments where the channels 401, 501, and the first and second ports 604, 606 may have a smooth or rounded edge at openings thereof, which serves to minimize or reduce trauma to bodily tissue, the channel or port openings may be provided with a smooth outer circumferential edge created by sanding the circumferential edges to a smooth finish or, for example, by coating the edges with a lubricious material.

In kit form, the present invention may be provided as a dilator having one or more channels where the dilator is capable of being used in conjunction with a plurality of types of elongated devices. The kit may further include a sheath and dilator pair configured for use together with any suitable elongated device. The elongated devices may include wires that form a guidewire, a wire-based needle, an RF needle (i.e., radiofrequency-based puncture device), a mechanical needle (i.e., mechanical-based puncture device), a catheter mechanism, a stylet mechanism, or any suitable device dimensioned for insertion into a dilator for accessing tissue of a patient.

The kit in accordance with the present invention may therefore include at least one tubular member and an elongated device operable to be combined to form an apparatus for coaxial fluid transfer between a proximal region of the apparatus and a distal region of the apparatus. The tubular member includes a tubular member lumen for receiving the elongated device. The tubular member is configured to include at least one channel that provides for fluid flow coaxial to the tubular member lumen. The channel(s) of the tubular member form a conduit for fluid in a region of the tubular member that is otherwise restricted to fluid flow when the elongated member is located in that region. In some embodiments of the kit, visual and/or tactile markers may be provided and configured for axially and/or rotationally aligning the channel(s) within the tubular member lumen.

It should be understood that the transverse cross-sectional shape of the tubular member(s) and elongated device may take any suitable configuration, and the invention is not limited in this regard. For example, the transverse cross-sectional shape of the resultant apparatus formed by the tubular member(s) and elongated device may be substantially circular, ovoid, oblong, or polygonal, among other possibilities. Furthermore, in some alternative embodiments that may be possible without straying from the intended scope of the present invention, the cross-sectional shape of the resultant apparatus may vary along the length thereof. For example, the cross-sectional shape of the proximal region may be substantially circular, while the cross-sectional shape of the distal region is substantially ovoid.

In all embodiments, the tubular member(s) and elongated device are made from suitable material that is biocompatible. As used herein, ‘biocompatible’ refers to a material that is suitable for use within the body during the course of a surgical procedure. Such materials include stainless steels, copper, titanium and nickel-titanium alloys (for example, NITINOL®), amongst others. Furthermore, in some embodiments, different regions of the tubular member(s) and elongated device are made from different materials such as, but not limited to, a nickel-titanium alloy such as NITINOL®, such that it provides flexibility to, for example, the distal region (e.g., the curved or shapeable section 102 at a distal region 101 when the invention is used in a system as seen in FIG. 1).

The embodiments of the invention described above are intended to be exemplary only. The scope of the invention is therefore intended to be limited solely by the scope of the appended claims. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. Although the invention has been described in conjunction with specific embodiments thereof, it is evident that many alternatives, modifications and variations are apparent to those skilled in the art. Accordingly, it is intended to embrace all such alternatives, modifications, and variations that fall within the scope of the appended claims.

What is claimed is:

1. An apparatus for fluid transfer, the apparatus comprising:
a tubular member having a circumferential sidewall, the circumferential sidewall including a tapered portion with an outer diameter that is tapered towards a distal tip of the tubular member;
a lumen for fluid transfer through the distal tip, the lumen centrally located within the circumferential sidewall; and
a channel coaxial with the lumen and located at least at a distal region of the tubular member;
wherein a fluid is movable through the channel irrespective of the presence of an elongated device located adjacent the channel.

2. The apparatus of claim 1, wherein the channel is located along a tapered portion of the tubular member.

3. The apparatus of claim 2, further including at least one additional channel.

4. The apparatus of claim 3, wherein each channel is separated from one another by an arc length around a periphery of the lumen.

5. The apparatus of claim 4, wherein the arc length separating each channel is equidistant.

6. The apparatus of claim 2, wherein the lumen is at least partially occluded by the elongated device located adjacent the channel.

7. The apparatus of claim 6, wherein the channel extends between a hub connected to the tubular member and the distal tip of the tubular member.

8. The apparatus of claim 7, wherein the tubular member is a dilator.

9. The apparatus of claim 8, wherein the channel is open to the lumen.

10. The apparatus of claim 3, further including an inner tubular member forming a dilator that is concentric to the tubular member which forms a sheath around the dilator, and the lumen is a sheath lumen.

11. The apparatus of claim 10, wherein the channels are formed within a distal section of the dilator.

12. The apparatus of claim 11, wherein each of the channels includes a proximal port in fluid communication with the sheath lumen and a distal port located at the distal tip of the dilator.

13. The apparatus of claim 12, wherein an interface between the sheath and the dilator forms seal by an interference fit.

14. The apparatus of claim 13, wherein fluid transfer through the channels is selectively enabled and disabled by relative linear movement between the sheath and the dilator that places the proximal port of each channel, respectively, into and out of fluid communication with the sheath lumen.

15. A kit forming an apparatus for fluid transfer, the kit comprising:
an elongated device capable of insertion into a tubular member for accessing tissue of a patient; and
a tubular member capable of accepting the elongated device, the tubular member having
a circumferential sidewall, the circumferential sidewall including a tapered portion with an outer diameter that is tapered towards a distal tip of the tubular member;
a lumen capable of fluid transfer through the distal tip, the lumen centrally located within the circumferential sidewall; and
a channel coaxial with the lumen and located at least at a distal region of the tubular member;
wherein, upon insertion of the elongated device into the tubular member, a fluid is capable of movement through the channel irrespective of the presence of the elongated device located adjacent the channel.

16. The kit of claim 15, wherein the tubular member forms a dilator including at least one additional channel.

17. The kit of claim 15, further including an inner tubular member capable of forming a dilator that is concentric to the tubular member, wherein the tubular member forms a sheath around the dilator, the lumen is a sheath lumen, and the dilator includes at least one additional channel.

18. The kit of claim 17, wherein the channels are formed within a distal section of the dilator, and the channels each includes a proximal port capable of fluid communication with the sheath lumen and a distal port located at the distal tip of the dilator.

19. The kit of claim 18, wherein an interface between the sheath and the dilator is capable of forming a seal by an interference fit.

20. The kit of claim 19, wherein fluid transfer through the channels is capable of being selectively enabled and disabled by relative linear movement between the sheath and the dilator that places the proximal port of each channel, respectively, into and out of fluid communication with the sheath lumen.
